(19) United States
(12) Patent Application Publication
(10) Pub. No.: US 2025/0268466 A1
(43) Pub. Date: Aug. 28, 2025

(54) COLOR VISION TEST WITH CONE ISOLATION AND CHROMATIC NOISE

(71) Applicant: EnChroma, Inc., Berkeley, CA (US)

(72) Inventors: Donald M. McPherson, Oakland, CA (US); Roger William Ellicock, JR., Berkeley, CA (US); Sunil K. Koovakkat, Hercules, CA (US)

(73) Assignee: EnChroma, Inc., Berkeley, CA (US)

(21) Appl. No.: 19/207,741

(22) Filed: May 14, 2025

Related U.S. Application Data
(63) Continuation of application No. PCT/US2023/082848, filed on Dec. 7, 2023.
(60) Provisional application No. 63/431,182, filed on Dec. 8, 2022.

(51) Int. Cl. A61B 3/06; A61B 3/00

(52) U.S. Cl. CPC A61B 3/066; A61B 3/0058

(57) ABSTRACT

The invention generally relates to a color vision test using pseudo-isochromatic plates to test color deficiency. The pseudo-isochromatic plates of the color vision test having a targeted cone class of the human eye (S-cone, M-cone, or L-cone) and incorporating chromatic noise.

### CROSS REFERENCE TO RELATED APPLICATIONS

This application is a continuation of International Patent Application No. PCT/US2023/082848 filed Dec. 7, 2023, which claims benefit of priority to U.S. Provisional Patent Application No. 63/431,182 filed Dec. 8, 2022. The foregoing applications are incorporated herein by reference in their entirety.

### FIELD OF THE INVENTION

The invention generally relates to color vision test using pseudo-isochromatic plates (PIP) to test color deficiency.

### BACKGROUND

The light that enters a normal human eye interacts with three wavelength sensitive cones and through phototransduction converts photons into neurochemical signals. These transduced cone signals are compared in midget retinal ganglion cells and relayed through the optic nerve for cortical processing. The three cone classes absorb photons across wavelengths covering the visible spectrum. Considering the wavelength range 400-700 nm, the S-cone absorbs photons over the wavelengths 400-500 nm with peak sensitivity 420-440 nm, the M-cone absorbs photons over the wavelengths 450-630 nm with peak sensitivity 534-555 nm and the L-cone absorbs photons over the wavelengths 500-700 nm with peak sensitivity 564-580 nm. Almost all illuminants and reflected light are broadband stimuli, engaging two or three cone classes to some degree.

Pseudo-isochromatic plates (PIP) have been used to test color deficiency. The most common PIPs are the Hardy Rand and Rittler Standard Pseudoisochromatic test (HRR), the Ishihara Pseudoisochromatic plate test and the Waggoner Pseudoisochromatic plate test. These PIPs mainly exist as tests printed on paper. Versions of PIP tests have been adapted for display on monitors but those test either require color calibration of the monitor and/or are not used as diagnostic tools. For example, The Rabin Cone Contrast Test (CCT) requires monitor calibration and administration and also does not account for individual differences in luminous response. A better color vision test for display on monitors is needed.

### SUMMARY

This specification discloses a color vision test that employs cone isolation and chromatic noise to mask error in color calibration of a monitor and differences in individual luminous response.

The color vision test is a series of pseudo-isochromatic plates (PIPs) having a targeted cone. Each PIP comprising at least three symbol swatches displayed on a monitor, each symbol swatch having a different color with LMS color coordinates, the symbol swatches having average symbol LMS color coordinates, each LMS coordinate of each symbol swatch is either substantially equal to the corresponding average symbol LMS color coordinate or within r distance of the corresponding average symbol LMS coordinate where 0<r<0.1 (chromatic noise), and at least one LMS color coordinate of each symbol swatch is within r distance from the corresponding average symbol LMS color coordinate. In addition, the PIP has at least three background swatches displayed on a monitor, each background swatch having a different color with LMS color coordinates, the background swatches having average background LMS color coordinates, each LMS coordinate of each background swatch is either substantially equal to the corresponding average background LMS color coordinate or within r distance of the corresponding average background LMS coordinate, and at least one LMS color coordinate of each background swatch is within r distance from the corresponding average background LMS color coordinate.

The PIP also has a percentage cone isolation of the average symbol LMS color coordinate to average background LMS color coordinate of the target cone greater than 85%. In some embodiments, r<0.05. In some embodiments, the background swatches have a grey color. In other embodiments, the background swatches has a non-grey color.

In some embodiments, the PIP targets the L cone. In some embodiments, the targeted cone is the M cone. In some embodiments, the targeted cone is the S cone.

In some embodiments, only one LMS color coordinate of each symbol swatch is within r distance from the corresponding average symbol LMS color coordinate. In other embodiments, two LMS color coordinates of each symbol swatch are within r distance from their corresponding average symbol LMS color coordinates. In other embodiments, all three LMS color coordinates of each symbol swatch are within r distance from their corresponding average symbol LMS color coordinates.

In some embodiments, the LMS color coordinate of each symbol swatch within r distance from the corresponding average symbol LMS color coordinate is a non-targeted cone LMS coordinate.

This specification discloses a color vision test comprising a series of pseudo-isochromatic plates including PIPs that target the L, M, and/or S cones.

This specification discloses a color vision test that can accurately test color vision even on a display monitor that has not been properly color calibrated.

### BRIEF DESCRIPTION OF THE DRAWINGS

FIG. 1 shows an exemplary pseudo-isochromatic plate (PIP).

FIG. 2 shows a plot of average CIE 1931 xy chromaticity coordinates for swatches for a series of PIP tests.

FIG. 3 is an enlargement of FIG. 2 showing a plot of the average CIE 1931 xy chromaticity coordinates for swatches for a series of PIP tests.

FIG. 4A shows a plot of CIE 1931 xy chromaticity coordinates for swatches in L cone isolation test. FIG. 4B shows a plot of CIE 1931 xy chromaticity coordinates for swatches in M cone isolation test. FIG. 4C shows a plot of CIE 1931 xy chromaticity coordinates for swatches in S cone isolation test.

FIG. 5 shows steps to calculate color coordinates for displaying swatches on a monitor.

FIG. 6 shows chromatic noise added to a background color.

FIG. 7 shows confusion lines within the Rec. 2020 display gamut where Tritan (S-cone). deutan (M-cone), and protan (L-cone) confusion lines not passing through a common neutral point, i.e. Equal Energy White (EEW) point.

### DETAILED DESCRIPTION

The following detailed description should be read with reference to the drawings, in which identical reference numbers refer to like elements throughout the different figures. The drawings, which are not necessarily to scale, depict selective embodiments and are not intended to limit the scope of the invention. The detailed description illustrates by way of example, not by way of limitation, the principles of the invention.

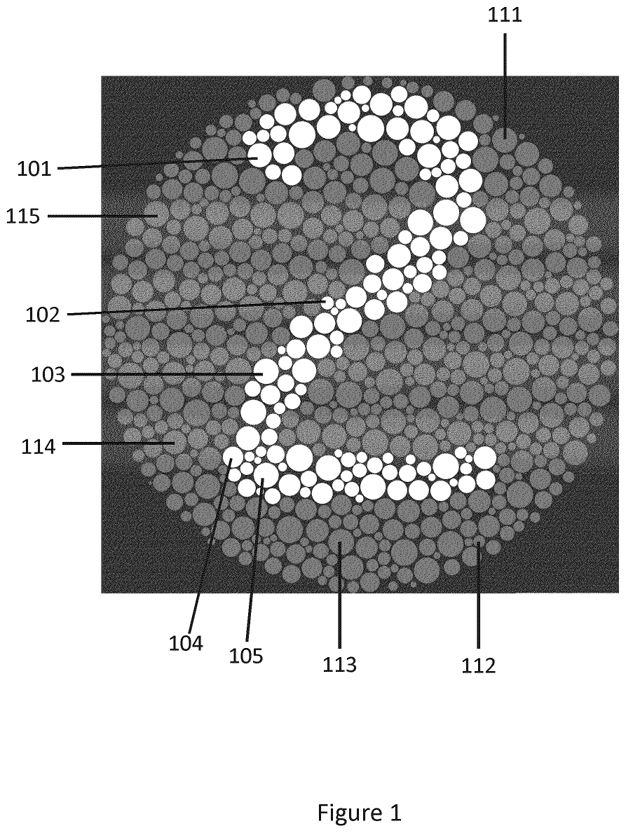

FIG. 1 shows a pseudo-isochromatic plate (PIP) test suitable for display on a monitor, e.g. a computer monitor, a desktop monitor, a smartphone display, or any electronic display device capable of displaying RGB values. The test can also be web-based and/or administered on-line or can be administered on premises. The test of FIG. 1 comprises many dots or swatches. The test includes symbol swatches 101, 102, 103, 104, 105 and background swatches 111, 112, 113, 114, 115. The symbol swatches together form a symbol, e.g. in FIG. 1 the number “2”. The background swatches are the non-symbol swatches. The test attempts to differentiate persons having normal color vision who can discern the difference between symbol and background swatches and persons having color vision deficiency (CVD) who cannot discern the difference between symbol and background swatches.

The test herein can be used to identify both hereditary and acquired color vision deficiency. The test can be used to monitor progression of loss of color vision. The test can be used to monitor improved performance in color vision after the individual wears special eyewear designed to improve their color contrast threshold. Further the test can be used with a filter placed between the test screen and the test subject, for instance by wearing tinted eyewear or tinted contact lenses.

It is possible to construct color stimuli that primarily activates a single cone class of the human eye (S-cone, M-cone, or L-cone) generally referred to herein as “cone isolation.” Using cone isolation allows evaluation of the isolated cone without engaging the non-targeted cones; in this manner color vision can be characterized as normal or defective, and more specifically color deficiency can be further defined by type and extent based on the identity of the defective cone and the severity of the defect. For example, a PIP test may be constructed so that persons with an S-cone deficiency would not be able to distinguish symbol from background, but a person with M-cone deficiency could. Such a test would be a S-cone isolation test.

Constructing a cone isolation test can be done with reference to confusion lines. Confusion lines are lines that can be drawn on the CIE 1931 color space. For two colors located on a confusion line, the colors may be a greater or lesser distance from each other along the line. In this specification, the distance between two colors along a confusion line is called cone contrast. Cone contrast can also be calculated in LMS color space as discussed below. Persons with CVD would confuse colors at a certain cone contrast, i.e. at a certain distance along a confusion line that a person with normal color vision would be able to distinguish.

The overall color vision test measures cone contrast sensitivity between a foreground symbol and the background in a series of test screens that change cone contrast along three confusion lines targeting the three cone classes. Each test screen targets a single cone class, with the non-targeted cone classes having very little cone contrast between the symbol and the background. To accommodate differences in displays the tests incorporate uniform random distributed chromatic noise across the symbol.

Different displays may represent given RGB values differently depending on variations in the display's primary chromaticities, gamma values, or other sources of error. This difference in display may cause a change in the relative stimulation of the cones, and cause errant contrast in the non-targeted cones. If this errant contrast is high enough, a color deficient observer could detect the symbol using their non-defective cones. Clinically administered tests solve this problem by frequently color calibrating the display upon which the test is administered. This color vision test described in this specification, instead of using color calibration, masks this non-targeted cone contrast by perturbing the color randomly (adding chromatic noise). As long as the errant contrast is approximately at or below the level of chromatic noise, it should be undetectable by the non-targeted cones, while preserving detectability by the targeted cone.

The color vision test described in this specification is a series of color test displayed on a screen, monitor, or device display. The display can be touch-screen, such as a tablet-type device or a smartphone. The first screen presents a landing page, where the test subject enters identifying information, such as name, email and phone number. The test subject also selects the viewing condition with respect to eyewear: no eyewear or color correcting eyewear. The test process is then explained on a new screen. The test commences with malingerer screens, whose purpose is to help the test subject understand the test process and to identify anyone intentionally failing the test. The test then presents a series of screens designed to examine the three cone classes, associated with the long-middle- and short-wavelength sensitive cones. Each of these three cones are examined using cone isolation test screens, with progressive change in cone contrast for the target cone relative to the background. The range of targeted cone contrasts relative to the background may vary from zero to largest amount achievable while remaining within the gamut of the display and maintaining cone isolation. The background may be chromatic or achromatic. After completion of the examination of the three cone classes, the test presents a report screen. The report screen shows scores for each cone class as a percent of normal color vision. The screen can present this information as a bar-type graph, as a percent score or both. The screen can also identify the test subject's color vision as to type and extent. The types may be termed normal, deuteranomalous, deutan defect, green-weak, protanomalous, protan defect, red-weak, tritanomalous, tritan defect, or blue-weak. The test subject can retake the test without tinted eyewear or while wearing tinted eyewear. Results are tracked with time to show performance change.

To calculate the appropriate color values for the symbol and background swatches, working in cone excitation space or (LMS) space is preferred, where L, M and S are the Long-Middle- and Short-wavelength sensitive cone sensitivity functions. The descriptor refers to the relative position of the cones' sensitivity in the visible portion of the spectrum of light, from wavelengths greater than ultraviolet (Short) to wavelengths less than infrared (Long).

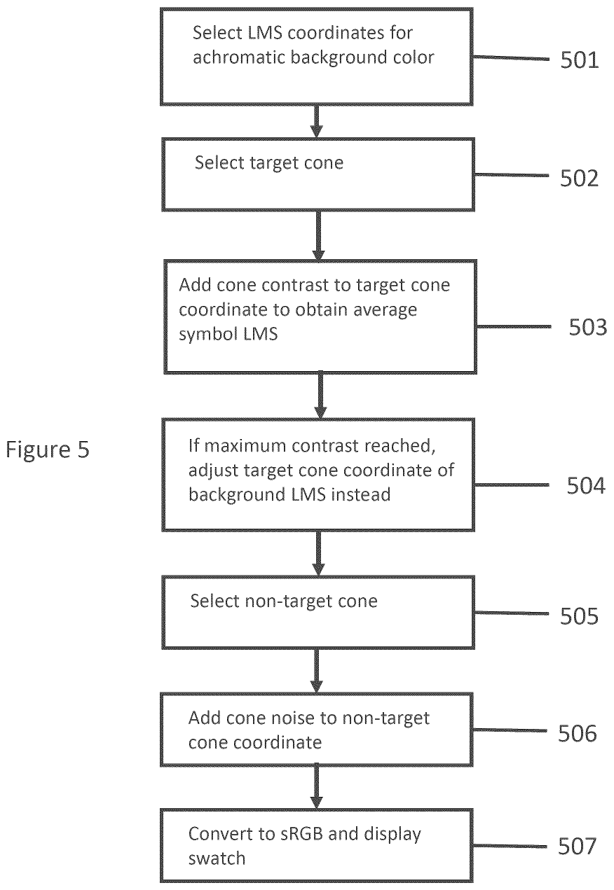

The steps to calculate the color values for the swatches in FIG. 1 are shown in FIG. 5. In step 501, an achromatic (grey) background color in LMS space is selected. The LMS value for the background is then

$${[ \begin{matrix}{L}_{grey} \\ {M}_{grey} \\ {S}_{grey}\end{matrix} ]}_{background}$$

Specific values may be (0.158, 0.135, 0.088) as used in Table 1. In step 502, a target cone, either the L, M, or S cones, is selected. For example, we select the L cone as the target cone. In step 503, cone contrast is added to the background grey color. We may choose the amount of cone contrast, C, to use, e.g. 10%, 20%, etc. The LMS coordinates for the symbol is calculated as follows:

$${[ \begin{matrix}L \\ M \\ S\end{matrix} ]}_{symbol} = [ \begin{matrix}( 1 + C ) * {L}_{grey} \\ {M}_{grey} \\ {S}_{grey}\end{matrix} ]$$

These calculated coordinates are average coordinate values. Table 1 shows the average LMS symbol and background coordinates for L and S cone tests calculated in this manner. For L cone, C values from about 13% to 70% were used. For S cone, C values from about 54% to 280% percent were used. The level of the test listed is Table 1 is an indication of the amount of cone contrast added. A level 1 test with the maximum amount of cone contrast added is easier for test users to pass then a level 16 test with the minimum amount of cone contrast added.

TABLE 1

| | | | Symbol | | | Background | | |
|---|---|---|---|---|---|---|---|---|
| Cone | Level | Contrast | L | M | S | L | M | S |
| L | 1 | 0.700 | 0.269 | 0.135 | 0.088 | 0.158 | 0.135 | 0.088 |
| L | 2 | 0.678 | 0.265 | 0.135 | 0.088 | 0.158 | 0.135 | 0.088 |
| L | 3 | 0.656 | 0.262 | 0.135 | 0.088 | 0.158 | 0.135 | 0.088 |
| L | 4 | 0.632 | 0.258 | 0.135 | 0.088 | 0.158 | 0.135 | 0.088 |
| L | 5 | 0.606 | 0.254 | 0.135 | 0.088 | 0.158 | 0.135 | 0.088 |
| L | 6 | 0.579 | 0.250 | 0.135 | 0.088 | 0.158 | 0.135 | 0.088 |
| L | 7 | 0.551 | 0.245 | 0.135 | 0.088 | 0.158 | 0.135 | 0.088 |
| L | 8 | 0.520 | 0.240 | 0.135 | 0.088 | 0.158 | 0.135 | 0.088 |
| L | 9 | 0.487 | 0.235 | 0.135 | 0.088 | 0.158 | 0.135 | 0.088 |
| L | 10 | 0.451 | 0.229 | 0.135 | 0.088 | 0.158 | 0.135 | 0.088 |
| L | 11 | 0.412 | 0.223 | 0.135 | 0.088 | 0.158 | 0.135 | 0.088 |
| L | 12 | 0.369 | 0.216 | 0.135 | 0.088 | 0.158 | 0.135 | 0.088 |
| L | 13 | 0.321 | 0.209 | 0.135 | 0.088 | 0.158 | 0.135 | 0.088 |
| L | 14 | 0.267 | 0.200 | 0.135 | 0.088 | 0.158 | 0.135 | 0.088 |
| L | 15 | 0.206 | 0.191 | 0.135 | 0.088 | 0.158 | 0.135 | 0.088 |
| L | 16 | 0.135 | 0.179 | 0.135 | 0.088 | 0.158 | 0.135 | 0.088 |
| M | 1 | 0.700 | 0.158 | 0.161 | 0.088 | 0.158 | 0.067 | 0.088 |
| M | 2 | 0.678 | 0.158 | 0.161 | 0.088 | 0.158 | 0.070 | 0.088 |
| M | 3 | 0.656 | 0.158 | 0.161 | 0.088 | 0.158 | 0.073 | 0.088 |
| M | 4 | 0.632 | 0.158 | 0.161 | 0.088 | 0.158 | 0.076 | 0.088 |
| M | 5 | 0.606 | 0.158 | 0.161 | 0.088 | 0.158 | 0.080 | 0.088 |
| M | 6 | 0.579 | 0.158 | 0.161 | 0.088 | 0.158 | 0.083 | 0.088 |
| M | 7 | 0.551 | 0.158 | 0.161 | 0.088 | 0.158 | 0.087 | 0.088 |
| M | 8 | 0.520 | 0.158 | 0.161 | 0.088 | 0.158 | 0.091 | 0.088 |
| M | 9 | 0.487 | 0.158 | 0.161 | 0.088 | 0.158 | 0.096 | 0.088 |
| M | 10 | 0.451 | 0.158 | 0.161 | 0.088 | 0.158 | 0.101 | 0.088 |
| M | 11 | 0.412 | 0.158 | 0.161 | 0.088 | 0.158 | 0.106 | 0.088 |
| M | 12 | 0.369 | 0.158 | 0.161 | 0.088 | 0.158 | 0.112 | 0.088 |
| M | 13 | 0.321 | 0.158 | 0.161 | 0.088 | 0.158 | 0.118 | 0.088 |
| M | 14 | 0.267 | 0.158 | 0.161 | 0.088 | 0.158 | 0.125 | 0.088 |
| M | 15 | 0.206 | 0.158 | 0.161 | 0.088 | 0.158 | 0.134 | 0.088 |
| M | 16 | 0.135 | 0.158 | 0.153 | 0.088 | 0.158 | 0.135 | 0.088 |
| S | 1 | 2.800 | 0.158 | 0.135 | 0.333 | 0.158 | 0.135 | 0.088 |
| S | 2 | 2.714 | 0.158 | 0.135 | 0.326 | 0.158 | 0.135 | 0.088 |
| S | 3 | 2.623 | 0.158 | 0.135 | 0.318 | 0.158 | 0.135 | 0.088 |
| S | 4 | 2.527 | 0.158 | 0.135 | 0.309 | 0.158 | 0.135 | 0.088 |
| S | 5 | 2.425 | 0.158 | 0.135 | 0.301 | 0.158 | 0.135 | 0.088 |
| S | 6 | 2.317 | 0.158 | 0.135 | 0.291 | 0.158 | 0.135 | 0.088 |
| S | 7 | 2.202 | 0.158 | 0.135 | 0.281 | 0.158 | 0.135 | 0.088 |
| S | 8 | 2.079 | 0.158 | 0.135 | 0.270 | 0.158 | 0.135 | 0.088 |
| S | 9 | 1.947 | 0.158 | 0.135 | 0.259 | 0.158 | 0.135 | 0.088 |
| S | 10 | 1.803 | 0.158 | 0.135 | 0.246 | 0.158 | 0.135 | 0.088 |
| S | 11 | 1.647 | 0.158 | 0.135 | 0.232 | 0.158 | 0.135 | 0.088 |
| S | 12 | 1.475 | 0.158 | 0.135 | 0.217 | 0.158 | 0.135 | 0.088 |
| S | 13 | 1.284 | 0.158 | 0.135 | 0.200 | 0.158 | 0.135 | 0.088 |
| S | 14 | 1.070 | 0.158 | 0.135 | 0.182 | 0.158 | 0.135 | 0.088 |
| S | 15 | 0.825 | 0.158 | 0.135 | 0.160 | 0.158 | 0.135 | 0.088 |
| S | 16 | 0.541 | 0.158 | 0.135 | 0.135 | 0.158 | 0.135 | 0.088 |

| | | | | | | | |
|---|---|---|---|---|---|---|---|
| Malingerer | — | 0.280 | 0.161 | 0.384 | 0.184 | 0.190 | 0.052 |

The LMS values for the M cone in Table 1 shows that a chromatic background was used instead of an achromatic background as was the case for the Land S cones. Chromatic backgrounds for cone isolation test may be used when varying the cone contrast by the desired amount would place the converted RGB value of that LMS value outside the RGB gamut of the display screen and thereby not allowing the display to render the color correctly. It is important that the colors generated by this test fall within the display gamut (sRGB, P3, Rec. 2020 color spaces) so that they may be accurately displayed on the screen. If any of the resultant RGB values fall above 255 or below 0, they will be clipped to 255 or 0, respectively, and this will make an inaccurate cone isolation test. Therefore, the display gamut imposes upper bounds on the level of cone contrast achievable if only achromatic backgrounds are used. However, it is possible to create higher cone contrasts while maintaining cone isolation by using a chromatic background.

In step 504, we ask whether the maximum contrast is reached, such that further increases in cone contrast would place the color outside the display gamut, which in this example is the sRGB gamut. If this is the case, we adjust the background color instead of the symbol color. If, for example, using a 20% cone contrast, C, puts the color at the edge of the sRGB gamut, we set the LMS value of the symbol at

$${[ \begin{matrix}L \\ M \\ S\end{matrix} ]}_{symbol} = [ \begin{matrix}{L}_{grey} \\ ( 1 + 0.2 ) * {M}_{grey} \\ {S}_{grey}\end{matrix} ]$$

Using this fixed LMS value for the symbol, we can calculate the LMS value of the background for a given value of cone contrast, C, as follow:

$${[ \begin{matrix}L \\ M \\ S\end{matrix} ]}_{background} = [ \begin{matrix}{L}_{grey} \\ ( 1 - ( C - 0.02 ) * {M}_{grey} \\ {S}_{grey}\end{matrix} ]$$

The adjustment of background color used for the M cone tests in table 1. At level 16 of the M cone test, a normal achromatic grey background is used. At level 15, the edge of the sRGB gamut is reached. For levels 1-14, the LMS coordinates of the symbol is the same as for level 15, but the LMS coordinates of the background varies in accordance with the above calculation.

Values in LMS space may be converted to XYZ coordinates in CIE color space coordinates using the transformation matrix given by Stockman and Sharpe (2000):

$${M}_{LMS \to XYZ} = [ \begin{matrix}1.94735469 & - 1.41445123 & 0.36476327 \\ 0.68990272 & 0.34 832189 & 0 \\ 0 & 0 & 1.93485343\end{matrix} ]$$

CIE XYZ color coordinates can further be converted to CIE xyY and standard display gamut sRGB values using transformations known to one of ordinary skill in the art. Table 2 gives the converted CIE xyY coordinates and sRGB values of the LMS values in Table 1. As discussed more below, the values in Table 2 are average sRGB values. The actual RGB values displayed in a test such as in FIG. 1 are obtained by adding chromatic noise as described below.

TABLE 2

| | | Symbol | | | Background | | | Symbol | | | Background | | |
|---|---|---|---|---|---|---|---|---|---|---|---|---|---|
| Cone | Level | R | G | B | R | G | B | x | y | Y | x | y | Y |
| L | 1 | 221 | 87 | 109 | 110 | 110 | 110 | 0.476 | 0.303 | 0.232 | 0.315 | 0.328 | 0.156 |
| L | 2 | 218 | 88 | 109 | 110 | 110 | 110 | 0.473 | 0.303 | 0.230 | 0.315 | 0.328 | 0.156 |
| L | 3 | 216 | 89 | 109 | 110 | 110 | 110 | 0.469 | 0.304 | 0.227 | 0.315 | 0.328 | 0.156 |
| L | 4 | 213 | 90 | 109 | 110 | 110 | 110 | 0.466 | 0.304 | 0.225 | 0.315 | 0.328 | 0.156 |
| L | 5 | 210 | 90 | 109 | 110 | 110 | 110 | 0.462 | 0.305 | 0.222 | 0.315 | 0.328 | 0.156 |
| L | 6 | 207 | 91 | 109 | 110 | 11 | 110 | 0.458 | 0.306 | 0.219 | 0.315 | 0.328 | 0.156 |
| L | 7 | 203 | 92 | 109 | 110 | 110 | 110 | 0.453 | 0.306 | 0.216 | 0.315 | 0.328 | 0.156 |
| L | 8 | 200 | 94 | 109 | 110 | 110 | 110 | 0.447 | 0.307 | 0.213 | 0.315 | 0.328 | 0.156 |
| L | 9 | 195 | 95 | 109 | 110 | 110 | 110 | 0.442 | 0.308 | 0.209 | 0.315 | 0.328 | 0.156 |
| L | 10 | 191 | 96 | 109 | 110 | 110 | 110 | 0.435 | 0.309 | 0.205 | 0.315 | 0.328 | 0.156 |
| L | 11 | 185 | 97 | 109 | 110 | 110 | 110 | 0.427 | 0.310 | 0.201 | 0.315 | 0.328 | 0.156 |
| L | 12 | 179 | 99 | 109 | 110 | 110 | 110 | 0.418 | 0.312 | 0.196 | 0.315 | 0.328 | 0.156 |
| L | 13 | 172 | 100 | 109 | 110 | 110 | 110 | 0.408 | 0.313 | 0.191 | 0.315 | 0.328 | 0.156 |
| L | 14 | 164 | 102 | 110 | 110 | 110 | 110 | 0.395 | 0.315 | 0.185 | 0.315 | 0.328 | 0.156 |
| L | 15 | 154 | 104 | 110 | 110 | 110 | 110 | 0.380 | 0.318 | 0.178 | 0.315 | 0.328 | 0.156 |
| L | 16 | 141 | 106 | 110 | 110 | 110 | 110 | 0.360 | 0.321 | 0.171 | 0.315 | 0.328 | 0.156 |
| M | 1 | 41 | 126 | 109 | 186 | 42 | 113 | 0.250 | 0.370 | 0.165 | 0.447 | 0.242 | 0.132 |
| M | 2 | 41 | 126 | 109 | 183 | 47 | 113 | 0.250 | 0.370 | 0.165 | 0.442 | 0.245 | 0.134 |
| M | 3 | 41 | 126 | 109 | 181 | 52 | 113 | 0.250 | 0.370 | 0.165 | 0.437 | 0.249 | 0.135 |
| M | 4 | 41 | 126 | 109 | 178 | 57 | 113 | 0.250 | 0.370 | 0.165 | 0.431 | 0.253 | 0.136 |
| M | 5 | 41 | 126 | 109 | 175 | 62 | 113 | 0.250 | 0.370 | 0.165 | 0.425 | 0.257 | 0.137 |
| M | 6 | 41 | 126 | 109 | 171 | 66 | 112 | 0.250 | 0.370 | 0.165 | 0.419 | 0.261 | 0.138 |
| M | 7 | 41 | 126 | 109 | 168 | 71 | 112 | 0.250 | 0.370 | 0.165 | 0.412 | 0.265 | 0.139 |
| M | 8 | 41 | 126 | 109 | 164 | 75 | 112 | 0.250 | 0.370 | 0.165 | 0.404 | 0.270 | 0.141 |
| M | 9 | 41 | 126 | 109 | 159 | 80 | 112 | 0.250 | 0.370 | 0.165 | 0.395 | 0.276 | 0.142 |
| M | 10 | 41 | 126 | 109 | 154 | 84 | 112 | 0.250 | 0.370 | 0.165 | 0.386 | 0.282 | 0.144 |
| M | 11 | 41 | 126 | 109 | 148 | 89 | 111 | 0.250 | 0.370 | 0.165 | 0.376 | 0.289 | 0.146 |
| M | 12 | 41 | 126 | 109 | 142 | 94 | 111 | 0.250 | 0.370 | 0.165 | 0.364 | 0.296 | 0.148 |
| M | 13 | 41 | 126 | 109 | 134 | 99 | 111 | 0.250 | 0.370 | 0.165 | 0.350 | 0.305 | 0.150 |
| M | 14 | 41 | 126 | 109 | 124 | 104 | 110 | 0.250 | 0.370 | 0.165 | 0.335 | 0.315 | 0.153 |
| M | 15 | 41 | 126 | 109 | 111 | 109 | 110 | 0.250 | 0.370 | 0.165 | 0.317 | 0.327 | 0.156 |
| M | 16 | 72 | 121 | 109 | 110 | 110 | 110 | 0.272 | 0.356 | 0.162 | 0.315 | 0.328 | 0.156 |
| S | 1 | 126 | 84 | 213 | 110 | 110 | 110 | 0.230 | 0.150 | 0.156 | 0.315 | 0.328 | 0.156 |
| S | 2 | 125 | 85 | 210 | 110 | 110 | 110 | 0.231 | 0.152 | 0.156 | 0.315 | 0.328 | 0.156 |
| S | 3 | 125 | 86 | 208 | 110 | 110 | 110 | 0.232 | 0.155 | 0.156 | 0.315 | 0.328 | 0.156 |
| S | 4 | 124 | 87 | 206 | 110 | 110 | 110 | 0.234 | 0.158 | 0.156 | 0.315 | 0.328 | 0.156 |
| S | 5 | 124 | 88 | 203 | 110 | 110 | 110 | 0.236 | 0.162 | 0.156 | 0.315 | 0.328 | 0.156 |
| S | 6 | 123 | 89 | 200 | 110 | 110 | 110 | 0.237 | 0.165 | 0.156 | 0.315 | 0.328 | 0.156 |
| S | 7 | 123 | 91 | 196 | 110 | 110 | 110 | 0.239 | 0.170 | 0.156 | 0.315 | 0.328 | 0.156 |
| S | 8 | 122 | 92 | 193 | 110 | 110 | 110 | 0.242 | 0.174 | 0.156 | 0.315 | 0.328 | 0.156 |
| S | 9 | 121 | 93 | 189 | 110 | 110 | 110 | 0.244 | 0.180 | 0.156 | 0.315 | 0.328 | 0.156 |
| S | 10 | 120 | 94 | 185 | 110 | 110 | 110 | 0.247 | 0.186 | 0.156 | 0.315 | 0.328 | 0.156 |
| S | 11 | 120 | 96 | 180 | 110 | 110 | 110 | 0.251 | 0.193 | 0.156 | 0.315 | 0.328 | 0.156 |
| S | 12 | 119 | 97 | 174 | 110 | 110 | 110 | 0.255 | 0.202 | 0.156 | 0.315 | 0.328 | 0.156 |
| S | 13 | 118 | 99 | 167 | 110 | 110 | 110 | 0.260 | 0.212 | 0.156 | 0.315 | 0.328 | 0.156 |
| S | 14 | 116 | 101 | 160 | 110 | 110 | 110 | 0.266 | 0.226 | 0.156 | 0.315 | 0.328 | 0.156 |
| S | 15 | 115 | 103 | 150 | 110 | 110 | 110 | 0.274 | 0.243 | 0.156 | 0.315 | 0.328 | 0.156 |
| S | 16 | 113 | 106 | 138 | 110 | 110 | 110 | 0.286 | 0.267 | 0.156 | 0.315 | 0.328 | 0.156 |

| | | | | | | | | | | | | |
|---|---|---|---|---|---|---|---|---|---|---|---|---|
| Malingerer | 219 | 70 | 226 | 0 | 140 | 76 | 0.315 | 0.172 | 0.249 | 0.268 | 0.482 | 0.193 |

CIE XYZ color coordinates may be converted to LMS values using the inverse of the Stockman and Sharpe matrix. This inverse matrix is:

$${M}_{XYZ \to LMS} = [ \begin{matrix}0.21057582 & 0.85509764 & - 0.0396983 \\ - 0.4170764 & 1.1772611 & 0.07862825 \\ 0 & 0 & 0.51683501\end{matrix} ]$$

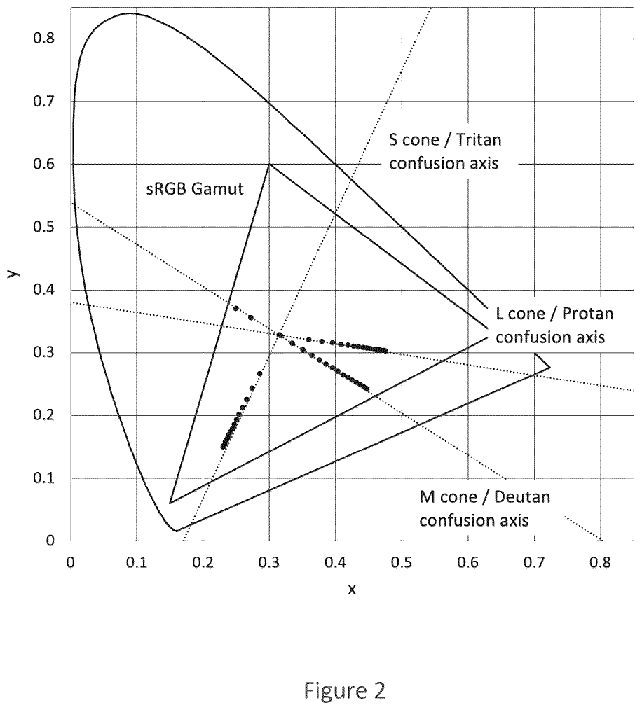

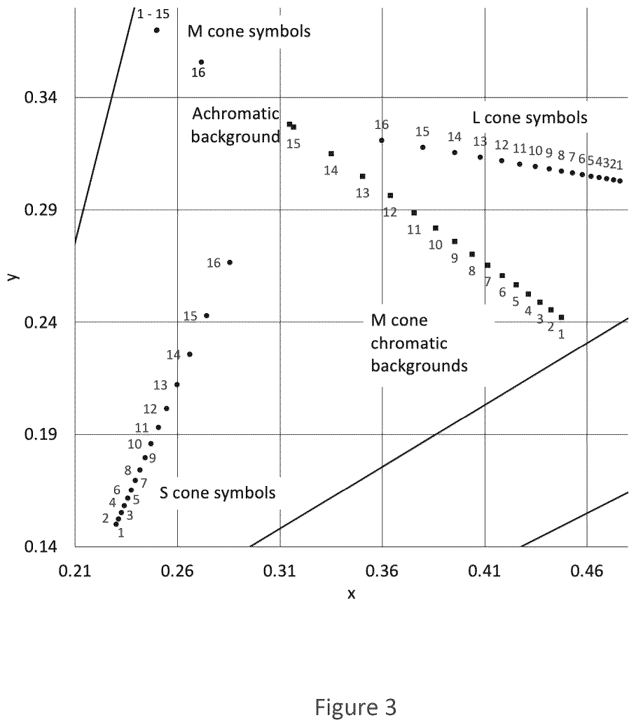

FIG. 2 plots the CIE 1931 xy chromaticity coordinates for each test screen given in Table 2. Note the luminance Y coordinate is not plotted. The confusion lines intersecting at an achromatic background point and the sRGB gamut is shown for reference. FIG. 3 shows a detailed plot of FIG. 2, with individual points labeled.

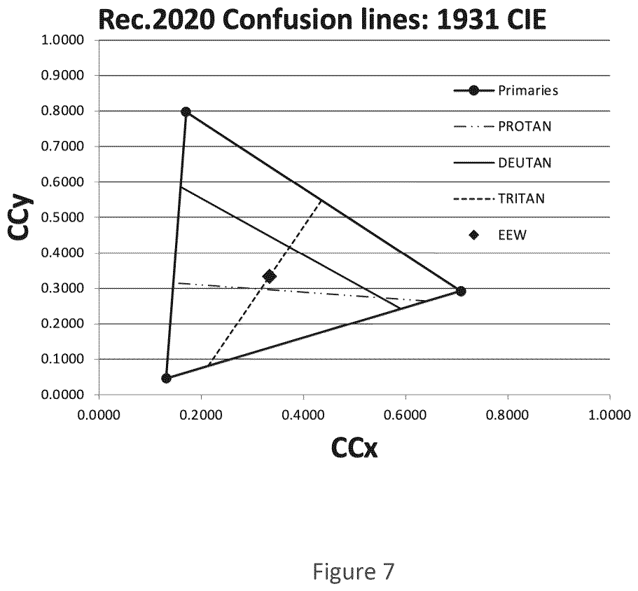

Although the above procedure calculates test color coordinates that correspond to confusion lines intersecting at a single achromatic (grey) point, the confusion lines for cone isolation test need not pass through a single achromatic (grey) point. A color vision test may be constructed using confusion lines that do not intersect at a single point. For example, one may use the confusion lines in FIG. 7 when constructing a color vision test in the Rec. 2020 color space. The confusion lines in FIG. 7 have longer line lengths and therefore larger contrast between foreground and background can be achieved. These confusion lines need not overlap but must not be collinear.

To obtain the actual RGB values used for the swatches, chromatic noise is added to the average LMS value in given in Table 1. To add chromatic noise, a random number, r, is added to the LMS coordinates given in Table 1. In some embodiments, a random number can be added to only one of the LMS coordinates. In some embodiments, a different random number is added to all three LMS coordinates. In other embodiments, a different random number is added to both the non-targeted cone coordinates. In other embodiments, a random non-targeted cone is chosen to which a random number is added. The random number is chosen within a range. For example, the amount of noise may be ±5%, i.e. a random number between −0.05 and +0.05 is chosen. Other amounts of randomness by be used. For example, r may range by ±10%, i.e. between −0.1 and +0.1. The amount of randomness used should be at level to mask the differences in displays and the error typically introduced by the lack of color calibration of the display.

Adding randomness in this manner will result in at least one LMS coordinate of every swatch being within a random distance away from the corresponding average LMS coordinate. The random distance is greater than zero but less than upper bound of randomness used, e.g. 5% or 10%. For example, at least one LMS coordinate of every symbol swatch will be within a 0.05 distance of the corresponding average LMS coordinate where distance is the difference between the LMS coordinate of the symbol swatch and the corresponding average LMS coordinate. LMS coordinates without added randomness will be substantially equal to the corresponding average LMS coordinate. The LMS coordinates will only be substantially equal to the corresponding average LMS coordinate because of measurement errors, rounding errors and sampling errors in calculating the average. For example, including more swatches in average calculation should reduce sampling errors.

In the embodiment of FIG. 5, to generate chromatic noise one random non-targeted cone is chosen to which a random number is added. The procedure for calculating color value of symbol swatches 101, 102, 103, 104, 105 and background swatches 111, 112, 113, 114, 115 is as follows. Depending on targeted cone, the cone contrast level, and whether the swatch is a symbol swatch or a background swatch, the appropriate average LMS is obtained from Table 1. In step 505, one of the non-target cones is selected at random. For example, if the target cone is the L cone, then one of the M and S cones is randomly selected.

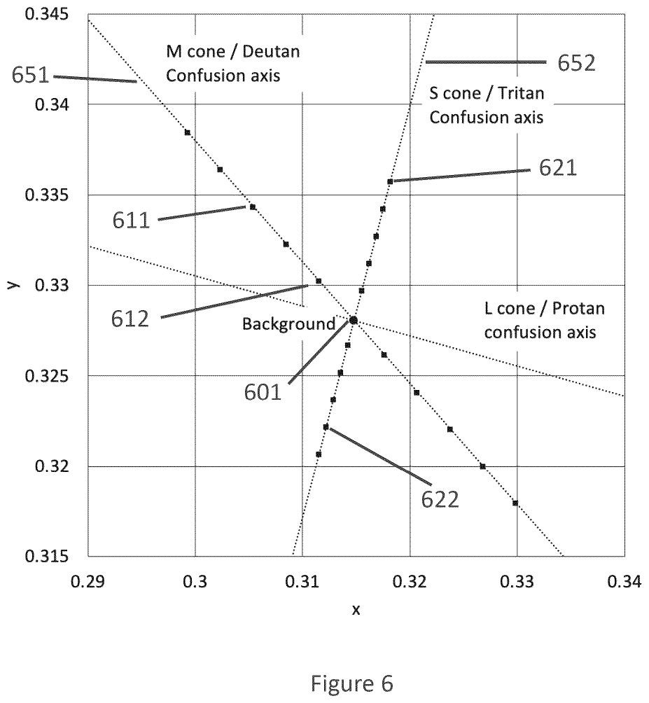

In step 506, chromatic noise is added to the non-target cone coordinate by adding a random number to the selected non-targeted cone coordinate. FIG. 6 shows an example of adding chromatic noise where the L cone is the targeted cone, e.g. in a L cone isolation test. Dot 601 indicates a color coordinate in some arbitrary color space. Line 651 is the M-cone confusion line in the color space that passes through dot 601 (e.g. the color coordinate for the background swatch). Line 652 is the S-cone confusion line in the color space that passes through dot 601. Dots 611 and 612 indicate the color coordinates of dot 601 after noise is added along the M-cone confusion line. Dots 621 and 622 indicate the color coordinate of dot 601 after noise is added along the S-cone confusion line.

The amount of noise added is a random number within a range. For example, the amount of noise may be ±5%, i.e. between −0.05 and +0.05. Other amounts of randomness by be chosen. For example, r may range by ±10%, i.e. between −0.1 and +0.1. To take a specific example in the LMS space in a L cone isolation test using cone contrast, C, where the M cone is the randomly chosen non-target cone for noise and r is the randomly chosen amount of noise, then LMS of the symbol swatch would be:

$${[ \begin{matrix}L \\ M \\ S\end{matrix} ]}_{symbol swatch} = [ \begin{matrix}( 1 + C ) * {L}_{grey} \\ ( 1 + r ) * {M}_{grey} \\ {S}_{grey}\end{matrix} ]$$

The LMS coordinates of symbol swatch may be converted into sRGB values for standard display gamuts (sRGB, P3, Rec. 2020) using the transformation matrices discussed above. For this specific example for a background swatch, using sRGB display gamut, if the S cone is the randomly chosen non-target cone, the LMS value of the background swatch may be

$${[ \begin{matrix}L \\ M \\ S\end{matrix} ]}_{background swatch} = [ \begin{matrix}{L}_{grey} \\ {M}_{grey} \\ ( 1 + r ) * {S}_{grey}\end{matrix} ]$$

Table 3 gives example LMS coordinates for 3 tests where cone noise is added to the LMS coordinates. For each test, 5 symbol swatch LMS coordinates are given and 5 background swatch LMS coordinates are given. For test 1, the average symbol LMS coordinates are (0.235, 0.135, 0.088) and the average background LMS coordinates are (0.158, 0.135, 0.088). For test 1, since the targeted cone is the L cone, the L coordinate of the average coordinate is not changed when adding noise, but one of the M or S coordinate is changed by a random amount. These are example coordinates are given in no particular order. A typical PIP test may a hundred or more of these coordinates, one coordinate set for each swatch.

TABLE 3

| | | Symbol | | | Background | | |
|---|---|---|---|---|---|---|---|
| Test | Targeted Cone | L | M | S | L | M | S |
| 1 | L | 0.235 | 0.137 | 0.088 | 0.158 | 0.142 | 0.088 |
| | L | 0.235 | 0.128 | 0.088 | 0.158 | 0.144 | 0.088 |
| | L | 0.235 | 0.135 | 0.080 | 0.158 | 0.135 | 0.071 |
| | L | 0.235 | 0.147 | 0.088 | 0.158 | 0.135 | 0.073 |
| | L | 0.235 | 0.135 | 0.107 | 0.158 | 0.135 | 0.095 |
| 2 | M | 0.158 | 0.161 | 0.092 | 0.163 | 0.096 | 0.088 |
| | M | 0.163 | 0.161 | 0.088 | 0.153 | 0.096 | 0.088 |
| | M | 0.158 | 0.161 | 0.112 | 0.158 | 0.096 | 0.114 |
| | M | 0.158 | 0.161 | 0.067 | 0.158 | 0.096 | 0.060 |
| | M | 0.159 | 0.161 | 0.088 | 0.158 | 0.096 | 0.084 |
| 3 | S | 0.158 | 0.142 | 0.259 | 0.165 | 0.135 | 0.088 |
| | S | 0.159 | 0.135 | 0.259 | 0.158 | 0.146 | 0.088 |
| | S | 0.158 | 0.129 | 0.259 | 0.158 | 0.135 | 0.088 |
| | S | 0.154 | 0.135 | 0.259 | 0.154 | 0.135 | 0.088 |
| | S | 0.157 | 0.135 | 0.259 | 0.158 | 0.142 | 0.088 |

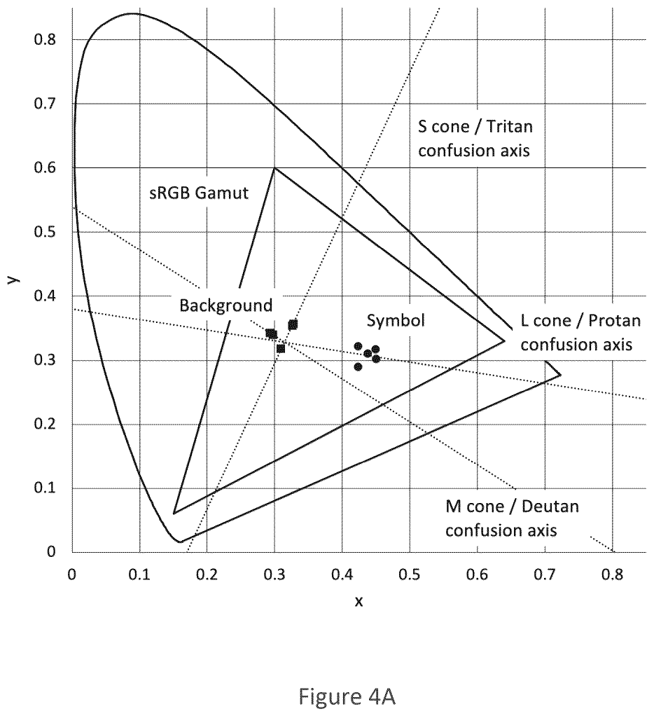

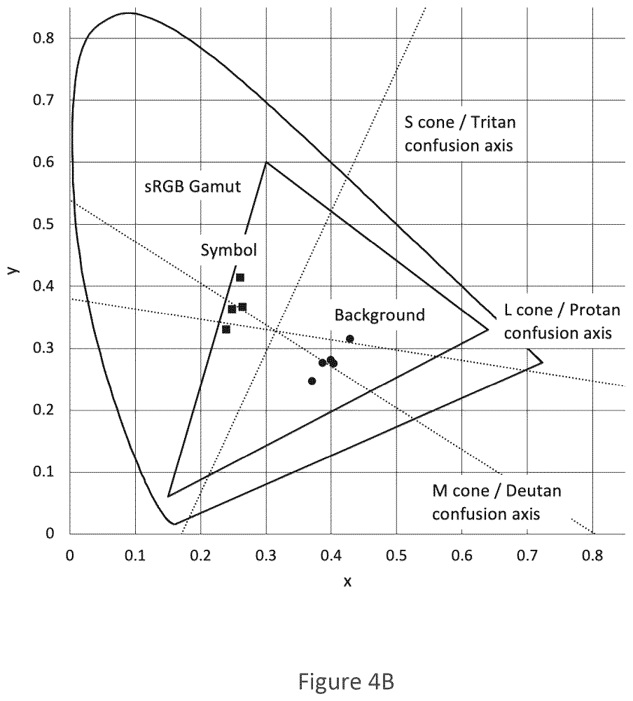

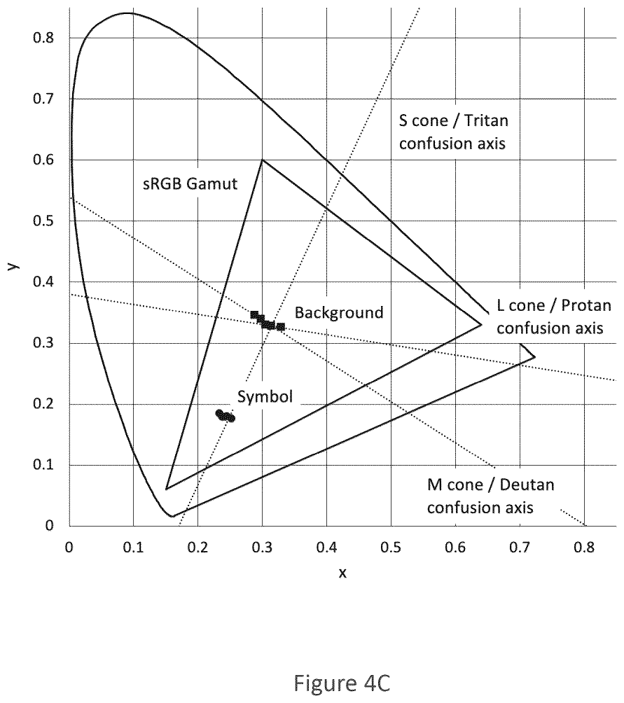

Table 4 gives the converted CIE 1931 xyY coordinates and sRGB values of the LMS values in Table 3. FIG. 4a is plot of the CIE 1931 xy chromaticity coordinates for an L cone isolating plate with added noise, as given in Table 3. FIG. 4b is a plot of the CIE 1931 xy chromaticity coordinates for an M cone isolating plate with added noise, as given in Table 3. FIG. 4c is a plot of the CIE 1931 xy chromaticity coordinates for an S cone isolating plate with added noise, as given in Table 3. In FIGS. 4a, 4b, and 4c, the sRGB gamut and the confusion lines are provided for reference. Note the Y coordinate is not plotted in FIGS. 4a, 4b and 4c.

TABLE 4

| | Targeted | Symbol | | | Background | | | Symbol | | | Background | | |
|---|---|---|---|---|---|---|---|---|---|---|---|---|---|
| Test | Cone | R | G | B | R | G | B | x | y | Y | x | y | Y |
| 1 | L | 194 | 96 | 109 | 97 | 115 | 110 | 0.439 | 0.310 | 0.159 | 0.298 | 0.339 | 0.210 |
| | L | 200 | 89 | 109 | 93 | 116 | 110 | 0.450 | 0.302 | 0.159 | 0.294 | 0.342 | 0.207 |
| | L | 195 | 96 | 103 | 109 | 111 | 98 | 0.450 | 0.317 | 0.156 | 0.328 | 0.356 | 0.209 |
| | L | 185 | 104 | 108 | 109 | 111 | 99 | 0.424 | 0.321 | 0.156 | 0.327 | 0.353 | 0.213 |
| | L | 196 | 93 | 122 | 110 | 109 | 115 | 0.424 | 0.289 | 0.156 | 0.310 | 0.317 | 0.209 |
| 2 | M | 42 | 126 | 111 | 164 | 78 | 112 | 0.248 | 0.363 | 0.146 | 0.404 | 0.276 | 0.165 |
| | M | 62 | 125 | 109 | 154 | 81 | 112 | 0.264 | 0.367 | 0.139 | 0.387 | 0.276 | 0.169 |
| | M | 46 | 124 | 124 | 160 | 76 | 128 | 0.240 | 0.330 | 0.142 | 0.370 | 0.247 | 0.165 |
| | M | 36 | 128 | 93 | 158 | 83 | 91 | 0.261 | 0.414 | 0.142 | 0.429 | 0.315 | 0.165 |
| | M | 45 | 126 | 109 | 159 | 80 | 109 | 0.252 | 0.370 | 0.142 | 0.399 | 0.280 | 0.166 |
| 3 | S | 109 | 99 | 189 | 120 | 109 | 110 | 0.234 | 0.184 | 0.160 | 0.329 | 0.326 | 0.159 |
| | S | 122 | 93 | 189 | 88 | 117 | 109 | 0.245 | 0.180 | 0.160 | 0.288 | 0.345 | 0.156 |
| | S | 130 | 88 | 189 | 109 | 110 | 110 | 0.252 | 0.176 | 0.156 | 0.314 | 0.328 | 0.154 |
| | S | 115 | 94 | 189 | 103 | 111 | 110 | 0.238 | 0.179 | 0.153 | 0.305 | 0.330 | 0.153 |
| | S | 120 | 93 | 189 | 97 | 115 | 110 | 0.243 | 0.179 | 0.158 | 0.298 | 0.339 | 0.155 |

Following the steps shown in FIG. 5, the color coordinates for all swatches in a PIP test such as in FIG. 1 can be determined. The PIP test of FIG. 1 exhibits cone isolation. This can be shown by taking the average value of background swatches and the average value of the symbol swatches and showing that the Weber contrast between symbol and background is primarily in one cone.

For example, the average RGB values of symbol swatches 101, 102, 103, 104, 105 can be obtained. The average RGB values of background swatches 111, 112, 113, 114, 115 can also be obtained. These average RGB values can be transformed into LMS coordinates using the transformation matrices discussed above and others known to those of ordinary skill in the art. Equivalently, the RGB value of the symbols and swatches can be first transformed into LMS coordinates and the average LMS coordinates obtained from those coordinates. If the background color is achromatic (i.e. grey), then using the average LMS coordinates, Weber contrasts for each LMS coordinate can be obtained as follows:

$$\begin{matrix}{L}_{w} = 100 ( \frac{{L}_{s} - {L}_{b}}{{L}_{b}} ) \\ {M}_{w} = 100 ( \frac{{M}_{s} - {M}_{b}}{{M}_{b}} ) \\ {S}_{w} = 100 ( \frac{{S}_{s} - {S}_{b}}{{S}_{b}} )\end{matrix}$$

where subscript w indicates Weber contrast, subscript s indicates average symbol coordinate value, and subscript b indicates average background coordinate value.

If the background swatches are colored, i.e. chromatic or non-grey, then the Weber contrasts for each LMS coordinate can be obtained as follows:

$$\begin{matrix}{L}_{w} = 100 ( \frac{{L}_{s} - {L}_{grey}}{{L}_{grey}} - \frac{{L}_{b} - {L}_{grey}}{{L}_{grey}} ) \\ {M}_{w} = 100 ( \frac{{M}_{s} - {M}_{grey}}{{M}_{grey}} - \frac{{M}_{b} - {M}_{grey}}{{M}_{grey}} ) \\ {S}_{w} = 100 ( \frac{{S}_{s} - {S}_{grey}}{{S}_{grey}} - \frac{{S}_{b} - {S}_{grey}}{{S}_{grey}} )\end{matrix}$$

where the LMS grey coordinates can be obtained by first calculating the average luminance of both symbol and background swatches in RGB and then obtaining the RGB grey color corresponding to that luminance value. The obtained RGB grey color can be transformed into LMS coordinates and then can used in the above equation.

From the Weber cone contrasts, the percentage cone isolation, subscript ci, can be obtained as follows:

$$\begin{matrix}{L}_{c i} = 1 \; 00 ( \vert {L}_{W} \vert / ( \vert {L}_{W} \vert + \vert {M}_{W} \vert + \vert \frac{{S}_{W}}{4} \vert ) ) \\ {M}_{c i} = 1 \; 00 ( \vert {M}_{W} \vert / ( \vert {L}_{W} \vert + \vert {M}_{W} \vert + \vert \frac{{S}_{W}}{4} \vert ) ) \\ {S}_{c i} = 1 \; 00 ( \vert \frac{{S}_{W}}{4} \vert / ( \vert {L}_{W} \vert + \vert {M}_{W} \vert + \vert \frac{{S}_{W}}{4} \vert ) )\end{matrix}$$

For a PIP test to exhibit cone isolation, generally the percentage cone isolation should be greater than 85%. For example, if the PIP test is a L-cone isolation test than the percentage cone isolation of the average symbol LMS to the average background LMS, L_{ci}, should be greater than 85%. As a further example, for a particular test, if L_{ci}=10%, M_{ci}=88%, and S_{ci}=2%, this would be a M-cone isolation test. The threshold for percentage cone isolation may be greater than 85% depending on whether error is introduced into the calculation via measurement, rounding errors and sampling errors in the average. In preferred embodiments, the percentage cone isolation is greater than 90%. In a most preferred embodiment, the percentage cone isolation is greater than 95%.

The color vision deficiency testing of this specification uses cone contrast within a single cone class. The isolated cone class is evaluated by changing the value of the cone contrast between the background and the symbol that is to be detected. In particular, this test does not require a calibrated display, instead relying on luminous and chromatic masking strategies by adding uniform randomly distributed chromatic noise across the symbol and background. The use of cone isolation and chromatic noise in a PIP test displayed on a monitor allows for accurate testing of person's color vision without the need for display monitor calibration.

In the color vision test, a series of screens with PIPs, such as in FIG. 1, are displayed on a device display. Every test user will have some threshold cone contrast value below which they cannot detect the symbol for each cone. For users with CVD, this threshold level is higher only for the user's defective cone. Rather than perform all 48 tests listed in table 1 on the user to determine the user's threshold level for each cone, the color vision test uses a binary search algorithm to quickly and accurately determine user's threshold levels. The idea is to test each cone in the middle of a range of contrasts. If the user passes, it indicates that their threshold is below that value, and if the user fails, their threshold is at or above that value. In this way we can progressively narrow the search range until we reach some predefined stopping point. If the stopping point is reached and the user has failed all plates, we test at the maximum contrast of the range.

The steps of the search algorithm are as follows:
1. Define a maximum and minimum contrast to search within, which can be specified to be within the display gamut of individual devices.
2. Define n, the number of levels to divide the contrast into. This must be a power of 2.
3. Set upper and lower to the maximum and minimum, respectively.
4. Test at contrast=(upper+lower)/2.
   a. If pass, set upper=(upper+lower)/2.
   b. If fail, set lower=(upper+lower)/2.
5. Repeat step 4 until (upper-lower)/2 is equal to (maximum-minimum)/n.
6. If all tests have been failed, test at contrast=maximum.
7. Result=100*(1−(lower−minimum)/(maximum−minimum)) %.
   a. If tested at maximum and failed, result=0%.

The result is formatted such that a perfect score=100%, and the subsequent scores decrease by steps of 100/n %. Since the lowest score this allows is 100/n % which is >0, the additional test at maximum contrast allows a score of 0%. Since there is a chance of errors or lucky guesses on individual tests, causing an incorrect result, the entire algorithm is repeated until the same result is reached twice. The test will then take, assuming no errors/lucky guesses, 2*log 2(n) plates to converge to a solution. Errors/lucky guess will make the test take longer.

The minimum and maximum contrast may be set such that the contrast range optimizes the resolution of the test while staying within the display gamut (sRGB, P3, Rec. 2020). Likewise, the contrast steps need not be a linear function of test levels, but may be exponential, logarithmic, or any other function, such that test resolution is optimized. In one embodiment of the test, the maximum contrast is equal to 0.7 for the L and M cones and 2.8 for the S cone, the minimum contrast is equal to 0.05 for the L and M cones and 0.2 for the S cone, and n is equal to 16. In this embodiment, the contrast steps are logarithmic functions of test level.

This algorithm is repeated for each cone class. The cones need not be tested sequentially; plates targeting the different cones may be interleaved. Additionally, a number of so-called “malingerer plates” may be added. These are identical in form to the usual test plates, but the symbol and background colors are chosen such that the symbol is detectable by any user, regardless of their color vision. The goal of these plates is to test whether the user understands how to take the test, and to ensure they are not deliberately attempting to fail the test. One embodiment of the test begins with two malinger plates, and then tests the L, M, and S cones sequentially, each following the algorithm described above.

In the color vision test, the test can be a static image showing one or more PIPs, such as in FIG. 1, displayed as a single image on a device display. Each PIP presents a symbol not detectable to a specific cone class, but only one cone class. The test user is asked to identify the symbols, with the answer screening to type of CVD. As an example, the test could be two screens presented on a single page, one for identifying deutan defects and one for identifying protan defects, each a PIP, such as in FIG. 1, with a different symbol.

This disclosure is illustrative and not limiting. Further modifications will be apparent to one skilled in the art in light of this disclosure and are intended to fall within the scope of the appended claims.

What is claimed is:

1. A pseudo-isochromatic plate having a targeted cone comprising:
at least three symbol swatches displayed on a monitor, each symbol swatch having a different color with LMS color coordinates, the symbol swatches having average symbol LMS color coordinates, each LMS coordinate of each symbol swatch is either substantially equal to the corresponding average symbol LMS color coordinate or within r distance of the corresponding average symbol LMS coordinate where 0<r<0.1, wherein at least one LMS color coordinate of each symbol swatch is within r distance from the corresponding average symbol LMS color coordinate; and
at least three background swatches displayed on a monitor, each background swatch having a different color with LMS color coordinates, the background swatches having average background LMS color coordinates, each LMS coordinate of each background swatch is either substantially equal to the corresponding average background LMS color coordinate or within r distance of the corresponding average background LMS coordinate, wherein at least one LMS color coordinate of each background swatch is within r distance from the corresponding average background LMS color coordinate;
the pseudo-isochromatic plate having a percentage cone isolation of the average symbol LMS color coordinate to average background LMS color coordinate of the target cone greater than 85%.

2. The pseudo-isochromatic plate of claim 1, wherein r<0.05.

3. The pseudo-isochromatic plate of claim 1, wherein at least one background swatch has a grey color.

4. The pseudo-isochromatic plate of claim 1, wherein at least one background swatch has a non-grey color.

5. The pseudo-isochromatic plate of claim 1, wherein the targeted cone is the L cone.

6. The pseudo-isochromatic plate of claim 1, wherein the targeted cone is the M cone.

7. The pseudo-isochromatic plate of claim 1, wherein the targeted cone is the S cone.

8. The pseudo-isochromatic plate of claim 1, wherein only one LMS color coordinate of each symbol swatch is within r distance from the corresponding average symbol LMS color coordinate and only one LMS color coordinate of each background swatch is within r distance from the corresponding average background LMS color coordinate.

9. The pseudo-isochromatic plate of claim 1, wherein two LMS color coordinates of each symbol swatch are within r distance from their corresponding average symbol LMS color coordinates and two LMS color coordinates of each background swatch is within r distance from the corresponding average background LMS color coordinates.

10. The pseudo-isochromatic plate of claim 1, wherein all three LMS color coordinates of each symbol swatch are within r distance from their corresponding average symbol LMS color coordinates and all three LMS color coordinates of each background swatch is within r distance from the corresponding average background LMS color coordinates.

11. The pseudo-isochromatic plate of claim 8, wherein the only one LMS color coordinate of each symbol and background swatch within r distance from the corresponding average LMS color coordinate is a non-targeted cone LMS coordinate.

12. The pseudo-isochromatic plate of claim 10, wherein the two LMS color coordinate of each symbol and background swatch within r distance from the corresponding average LMS color coordinate are non-targeted cone LMS coordinates.

13. A color vision test comprising a plurality of pseudo-isochromatic plates of claim 1.

14. A color vision test comprising a plurality of pseudo-isochromatic plates of claim 1 targeting the L cone; a plurality of pseudo-isochromatic plates of claim 1 targeting the M cone; and a plurality of pseudo-isochromatic plates of claim 1 targeting the S cone.

15. The color vision test of claim 13, wherein the plurality of pseudo-isochromatic plates are displayed on a monitor that is not properly color calibrated.
